(19) United States
(12) Patent Application Publication
(10) Pub. No.: US 2025/0069221 A1
(43) Pub. Date: Feb. 27, 2025

(54) MEDICAL IMAGE PROCESSING APPARATUS AND MEDICAL IMAGE PROCESSING PROGRAM

(71) Applicant: FUJIFILM Healthcare Corporation, Chiba (JP)

(72) Inventor: TOMOAKI CHONO, Chiba (JP)

(73) Assignee: FUJIFILM Healthcare Corporation, Chiba (JP)

(21) Appl. No.: 18/764,120

(22) Filed: Jul. 3, 2024

(30) Foreign Application Priority Data
Aug. 25, 2023 (JP) 2023-137455

(51) Int. Cl. G06T 7/00; A61B 8/08; G16H 50/20

(52) U.S. Cl. CPC G06T 7/0012; A61B 8/0883; G16H 50/20; G06T 2200/24; G06T 2207/10132; G06T 2207/20081; G06T 2207/20092; G06T 2207/30048

(57) ABSTRACT

A learning model is trained to output output data indicating an inference result regarding an ultrasound image that is a medical image, based on input data representing the ultrasound image. An inference parameter change unit changes an inference parameter, which is a parameter that is an adjustment target in training processing of the learning model and that affects the output data, in response to an instruction from a user. An inference processing unit outputs an inference result regarding a target ultrasound image, which is an inference processing target, based on output data in a case in which the target ultrasound image is input to the learning model having the changed inference parameter.

### CROSS-REFERENCE TO RELATED APPLICATION

This application claims the priority benefits of Japanese application no. 2023-137455, filed on Aug. 25, 2023. The entirety of the above-mentioned patent application is hereby incorporated by reference herein and made a part of this specification.

### BACKGROUND

### 1. Technical Field

The present specification discloses improvements of a medical image processing apparatus and a medical image processing program.

### 2. Description of the Related Art

A medical image, which is an image representing a structure or a function inside a subject, is mainly used for diagnosing or treating a disease. Examples of the medical image include an X-ray image, a computer tomography (CT) image, a nuclear magnetic resonance image (MRI), and an ultrasound image. On the other hand, a (machine) learning model is known in which internal parameters are adjusted by performing training processing using training data. The learning model that has been sufficiently trained can predict (infer) data corresponding to each of various input data with high accuracy, to output an inference result as output data.

In the related art, it has been proposed to incorporate a learning model into a medical image processing apparatus for processing the medical image.

For example, JP2020-92739A discloses an ultrasound image forming apparatus that constructs a trained model that learns a correlation between ultrasound image data and an evaluation value of the ultrasound image data, and changes a parameter related to the acquisition of the ultrasound image data in a case in which the evaluation value output by the trained model that receives, as input, the ultrasound image data to be processed is less than a threshold value.

### SUMMARY

Meanwhile, it is considered to train the learning model to output the output data indicating the inference result regarding the medical image. Examples of the learning model that outputs the inference result regarding the medical image include a learning model that infers a contour line of (an interior cavity of) a heart in a case in which the heart is shown in the medical image.

In this case, the learning model is trained using a large number of medical images as the training data, and the internal parameters (referred to as “inference parameters” in the present specification) are set. However, there is a case in which the inference result of the trained learning model does not completely match a result requested by a specific user. For example, in the above-described example, there is a case in which a certain user wants to know a rough contour line of the heart, but the learning model outputs the contour line of the heart in a considerably fine manner, and thus the smoothness is lost.

An object of a medical image processing apparatus disclosed in the present specification is to provide a medical image processing apparatus that can bring an inference result using a learning model close to a user's request in a case in which an inference on a medical image is performed by using the learning model.

The present specification discloses a medical image processing apparatus comprising: a learning model that has been trained to output, based on input data representing a medical image, output data indicating an inference result regarding the medical image; an inference parameter change unit that changes an inference parameter, which is a parameter that is an adjustment target in training processing of the learning model and that affects the output data, in response to an instruction from a user; and an inference processing unit that outputs an inference result regarding a target medical image, which is a medical image that is an inference processing target, based on output data in a case in which the target medical image is input to the learning model having the changed inference parameter.

The medical image processing apparatus may further comprise: a display controller that displays the target medical image and the inference result regarding the target medical image on a display, and changes a display content of the inference result on the display in response to the change of the inference parameter.

In a case in which the user makes a correction to the inference result regarding the target medical image displayed on the display, the inference parameter change unit may change the inference parameter based on the corrected inference result.

The medical image processing apparatus may further comprise: a parameter change history database that stores the changed inference parameter that is changed in response to the instruction from the user, in which the inference parameter change unit changes the inference parameter based on the changed inference parameter stored in the parameter change history database, without depending on the instruction from the user in a case in which inference processing on the target medical image is performed in response to the instruction from the user.

The parameter change history database may store a user identifier for identifying the user who gives the instruction to change the inference parameter and the changed inference parameter that is changed in response to the instruction from the user in association with each other, and in a case in which the inference parameter related to the user is stored in the parameter change history database, the inference parameter change unit may change the inference parameter based on the changed inference parameter associated with the user identifier of the user in the parameter change history database, without depending on the instruction from the user in a case in which the inference processing on the target medical image is performed in response to the instruction from the user.

The medical image may be an ultrasound image formed by irradiating a heart of a subject with ultrasound, and the inference processing unit may output, as the inference result regarding the target medical image, information used for measurement of the heart using the ultrasound image that is the target medical image.

The present specification discloses a medical image processing program causing a computer to function as: a learning model that has been trained to output, based on input data representing a medical image, output data indicating an inference result regarding the medical image; an inference parameter change unit that changes an inference parameter, which is a parameter that is an adjustment target in training processing of the learning model and that affects the output data, in response to an instruction from a user; and an inference processing unit that outputs an inference result regarding a target medical image, which is a medical image that is an inference processing target, based on output data in a case in which the target medical image is input to the learning model having the changed inference parameter.

With the medical image processing apparatus disclosed in the present specification, it is possible to bring the inference result using the learning model close to the user's request in a case in which the inference on the medical image is performed by using the learning model.

### BRIEF DESCRIPTION OF THE DRAWINGS

FIG. 1 is a schematic configuration diagram of an ultrasound diagnostic apparatus according to the present embodiment.

FIG. 2 is a diagram showing a display example of an inference result regarding a target ultrasound image.

FIG. 3 is a diagram showing an example of a change screen of inference parameters.

FIG. 4 is a diagram showing a display example of an inference result using the changed inference parameters.

FIG. 5 is a conceptual diagram showing an example of contents of a parameter change history DB.

FIG. 6 is a flowchart showing a flow of processing of an ultrasound diagnostic apparatus according to the present embodiment.

### DESCRIPTION OF THE EMBODIMENTS

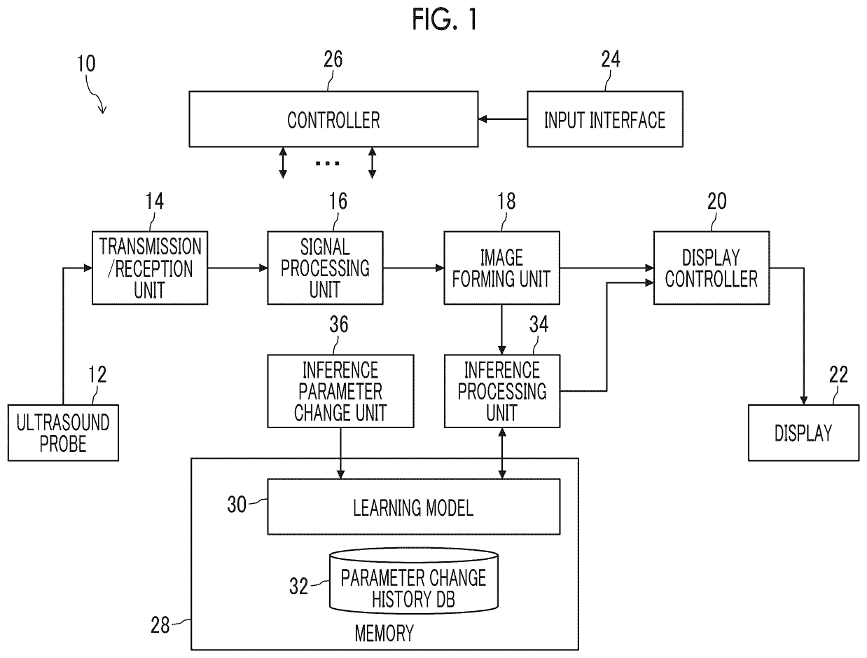

FIG. 1 is a schematic configuration diagram of an ultrasound diagnostic apparatus 10 as a medical image processing apparatus according to the present embodiment. The ultrasound diagnostic apparatus 10 is a medical apparatus installed in a medical institution, such as a hospital.

The ultrasound diagnostic apparatus 10 is an apparatus that scans a subject with an ultrasound beam to generate an ultrasound image as a medical image based on a reception signal obtained by the scanning. In particular, in the present embodiment, the ultrasound diagnostic apparatus 10 forms an ultrasound tomographic image (B-mode image) in which an amplitude intensity of reflected waves from a scanning surface is converted into the brightness based on the reception signal. It should be noted that the ultrasound diagnostic apparatus 10 can also form other ultrasound images, such as a Doppler image formed based on a difference (Doppler shift) in frequency between transmitted waves and received waves and representing a motion velocity of a tissue in the subject.

It should be noted that a transmission/reception unit 14, a signal processing unit 16, an image forming unit 18, a display controller 20, an inference processing unit 34, and an inference parameter change unit 36 provided in the ultrasound diagnostic apparatus 10 are configured by a processor. The processor includes at least one of a general-purpose processing apparatus (for example, a central processing unit (CPU)) or a dedicated processing apparatus (for example, a graphics processing unit (GPU), an application specific integrated circuit (ASIC), a field-programmable gate array (FPGA), and a programmable logic device). The processor may be configured by the cooperation of a plurality of processing apparatuses existing at physically separated positions, instead of being configured by one processing apparatus. In addition, each of the above-described units may be realized by a cooperation between hardware, such as the processor, and software.

An ultrasound probe 12 is a device that transmits and receives the ultrasound to and from the subject. The ultrasound probe 12 has an oscillation element array including a plurality of oscillation elements that transmit and receive the ultrasound to and from the subject.

The transmission/reception unit 14 transmits a transmission signal to the ultrasound probe 12 (specifically, each oscillation element of the oscillation element array) in response to the control of a controller 26 (described later). As a result, the ultrasound is transmitted from each oscillation element toward the subject.

In addition, the transmission/reception unit 14 receives the reception signal from each oscillation element that receives the reflected waves from the subject. The transmission/reception unit 14 includes an adder and a plurality of delayers corresponding to the respective oscillation elements, and phase adjustment addition processing of aligning and adding phases of the reception signals from the respective oscillation elements is performed by the adder and the plurality of delayers. As a result, a reception beam signal in which information indicating the signal intensity of the reflected waves from the subject is arranged in a depth direction of the subject is formed.

The signal processing unit 16 executes various types of signal processing including filter processing, such as applying a bandpass filter, detection processing, and the like on the reception beam signal from the transmission/reception unit 14.

The image forming unit 18 forms the ultrasound tomographic image (B-mode image) based on the reception beam signal subjected to the signal processing by the signal processing unit 16. First, the image forming unit 18 converts the reception beam signal into data in a coordinate space of the ultrasound image. Then, the image forming unit 18 forms the ultrasound tomographic image based on the coordinate conversion signal.

The display controller 20 executes control of displaying, on a display 22, the ultrasound tomographic image formed by the image forming unit 18 and various types of other information. The display 22 is, for example, a display device configured by a liquid crystal display, an organic electro luminescence (EL), or the like.

An input interface 24 is configured by, for example, a button, a track ball, and a touch panel. The input interface 24 is used to input a command from a user to the ultrasound diagnostic apparatus 10. In the present embodiment, the display 22 is a touch panel, and the display 22 also exerts a function of the input interface 24.

The controller 26 includes at least one of a general-purpose processor (for example, a central processing unit (CPU)) or a dedicated processor (for example, a graphics processing unit (GPU), an application specific integrated circuit (ASIC), a field-programmable gate array (FPGA), and a programmable logic device). The controller 26 may be configured by the cooperation of a plurality of processing apparatuses existing at physically separated positions, instead of being configured by one processing apparatus. The controller 26 controls each unit of the ultrasound diagnostic apparatus 10 in accordance with a medical image processing program stored in a memory 28 described later.

The memory 28 includes a hard disk drive (HDD), a solid state drive (SSD), an embedded multi media card (eMMC), a read only memory (ROM), or the like. The memory 28 stores the medical image processing program for operating each unit of the ultrasound diagnostic apparatus 10. It should be noted that the medical image processing program can also be stored, for example, in a computer-readable non-transitory storage medium, such as a universal serial bus (USB) memory or a CD-ROM. The ultrasound diagnostic apparatus 10 can read the medical image processing program from such a storage medium to execute the medical image processing program.

As shown in FIG. 1, a learning model 30 is stored in the memory 28. The learning model 30 is a model that uses, as input data, data representing the medical image to perform an inference on the medical image related to the input data, and outputs an inference result. In the present specification, an example will be described in which the learning model 30 receives, as input, the ultrasound tomographic image representing the cross section of the heart, and outputs, as the inference result, information used for the measurement of the heart using the ultrasound tomographic image. In particular, in the present specification, an example will be described in which the learning model 30 infers a contour line of the heart and a feature position (for example, a cardiac apex position, a valve annulus position, or the like) of the heart, as the information used for the measurement of the heart, and outputs the inference result. The input data representing the medical image to be input to the learning model 30 is not limited to the medical image itself. For example, in a case in which the medical image is the ultrasound image, the reception beam signal before the ultrasound image formation may be used. The medical image is not limited to the ultrasound image, and may be, for example, an X-ray image, a CT image, or an MRI. Further, the inference content of the learning model 30 may be any content as long as the inference content is related to the input medical image.

The learning model 30 is, for example, a model using an active shape model (ASM). The ASM is a model that represents a target shape (in the present embodiment, the contour line of the heart) by a set of points. The ASM incorporated in the learning model 30 in the present embodiment calculates an average shape of a plurality of contour lines of the heart as the training data, and performs a principal component analysis of the plurality of contour lines of the heart, to acquire a plurality of combinations of eigenvalues and eigenvectors of the contour lines. Thereafter, a weight is added to each eigenvector. By changing the weight of each eigenvector, the shape of the contour line of the heart is changed. For example, by increasing a weight of a high-rank principal component (for example, a first principal component) and decreasing a weight of a low-rank principal component, the learning model 30 infers a rough shape of the contour line of the heart (in other words, the contour line of the heart is smoothed). On the other hand, by decreasing the weight of the high-rank principal component and increasing the weight of the low-rank principal component, the learning model 30 enhances the details of the contour line of the heart and performs the inference. In this way, the weight of each eigenvector is a parameter that affects the output data of the learning model 30.

The weight of each eigenvector in the ASM is a parameter that is an adjustment target in the training processing of the learning model 30. The learning model 30 uses, as the training data, a combination of the ultrasound image representing the heart and the contour line of the heart in the ultrasound image, and adjusts inference parameters including the weight of each eigenvector in the ASM described above such that the contour line or the feature position of the heart represented in the ultrasound image can be inferred. It should be noted that the inference parameters in the present specification mean parameters that are adjustment targets in the training processing of the learning model 30 and that affect the inference result of the learning model 30 (that is, the output data of the learning model 30). The learning model 30 that has been sufficiently trained can output the contour line and the feature position of the heart in the ultrasound image with high accuracy based on the ultrasound image representing the heart.

Further, as shown in FIG. 1, the memory 28 stores a parameter change history database (DB) 32. The details or the use method of the parameter change history DB 32 will be described later together with the details of the inference parameter change unit 36.

The inference processing unit 34 performs inference processing on the target medical image (target ultrasound image in the present embodiment) which is the medical image that is an inference processing target, and outputs the inference result. Specifically, the inference processing unit 34 inputs the target ultrasound image to the learning model 30 that has been trained, and outputs the inference result regarding the target ultrasound image based on the output data of the learning model 30 with respect to the input data. In the present embodiment, as described above, the inference processing unit 34 infers the contour line and the feature position of the heart for the target ultrasound image representing the heart, and outputs the inference result.

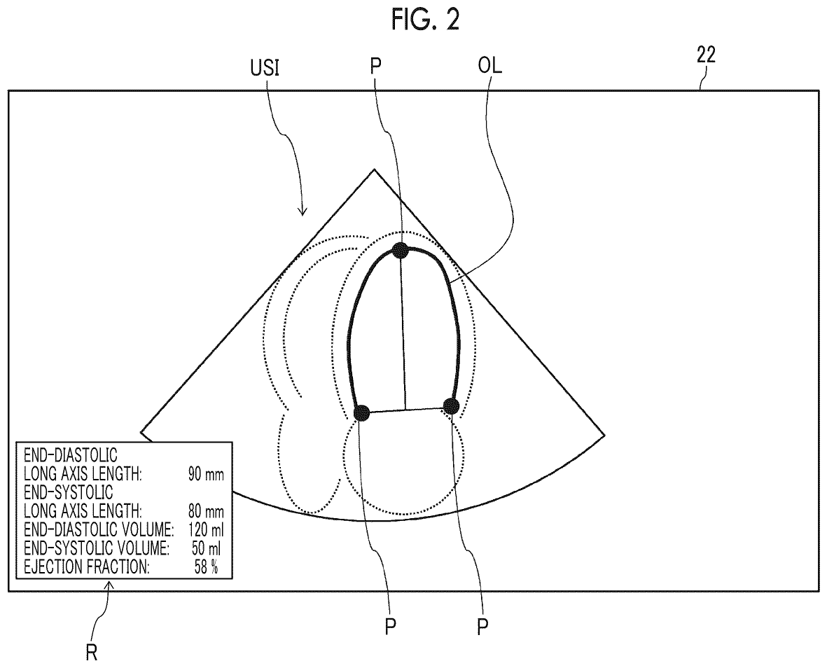

The display controller 20 displays, on the display 22, the inference result of the inference processing unit 34 related to the target ultrasound image. FIG. 2 is a diagram showing a display example of an inference result regarding a target ultrasound image USI. The target ultrasound image USI shown in FIG. 2 shows a four-chamber cross section of the heart, and as the inference result regarding the target ultrasound image USI, a contour line OL and a feature position P of the heart (particularly, a left ventricle) are shown.

It should be noted that the controller 26 may have a function as a measurement unit, and may perform the measurement on the heart represented in the target ultrasound image USI based on at least one of the contour line OL or the feature position P inferred by the inference processing unit 34. For example, the controller 26 may measure an end-diastolic long axis length, an end-systolic long axis length, an end-diastolic volume, an end-systolic volume, an ejection fraction, and the like of the heart. The display controller 20 may display a measurement result R of the controller 26 on the display 22 along with the inference result of the inference processing unit 34.

The inference parameter change unit 36 changes the inference parameters of the learning model 30 in response to an instruction from the user. As described above, the inference parameters of the learning model 30 for performing the inference regarding the target ultrasound image are adjusted by the training processing, but in the present embodiment, the inference parameter change unit 36 can change at least a part of the inference parameters of the learning model 30 in response to the instruction from the user. In other words, the user can change at least a part of the inference parameters of the learning model 30. As described above, since the inference parameters are the parameters that affect the inference result of the learning model 30, in a case in which the inference parameter change unit 36 changes the inference parameters, the inference result of the learning model 30 is changed.

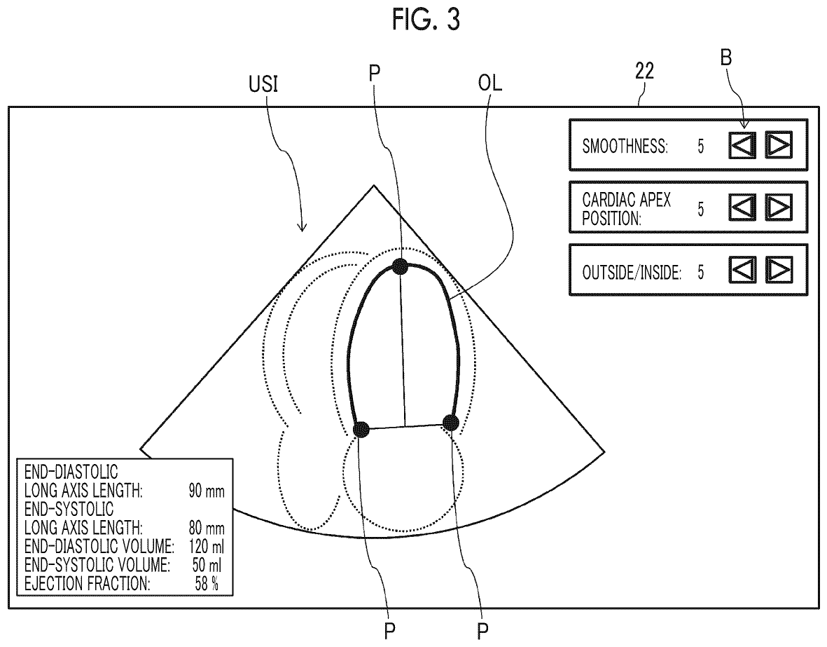

FIG. 3 is a diagram showing an example of a change screen of the inference parameters. In the present embodiment, the display controller 20 displays a button B for the user to change the inference parameters on the display 22, which is the touch panel, along with the inference result of the inference processing unit 34. The user can operate the button B to input the instruction to change the inference parameters of the learning model 30 to the inference parameter change unit 36. The inference parameter change unit 36 changes the inference parameters of the learning model 30 in response to the input change instruction.

The learning model 30 generally has a plurality of inference parameters, and there may be a case in which it is difficult for the user to understand which inference parameter affect the inference result of the learning model 30 and how the inference parameter affect the inference result. Therefore, inference parameter information in which the inference parameters of the learning model 30 are associated with items of the inference result of the learning model 30 affected by the inference parameters may be stored in the memory 28 in advance, and the display controller 20 may display the item included in the inference parameter information and the button B for changing the inference parameter corresponding to the item on the display 22 in association with each other.

For example, in the example of FIG. 3, the display controller 20 displays the button B for an item “smoothness (of the contour line OL of the heart)”, the button B for an item “cardiac apex position”, and the button B for an item “outside/inside”. The button B for each item is configured by a set of the button B (in the example of FIG. 3, the button B indicated by a leftward arrow) for changing the inference parameter for the item in a first direction (for example, a decreasing direction) and the button B (in the example of FIG. 3, the button B indicated by a rightward arrow) for changing the inference parameter for the item in a second direction (for example, an increasing direction) opposite to the first direction.

Further, the display controller 20 may display an index value (in the example of FIG. 3, the index value of each item is “5”) representing a current value of the inference parameter of each item. The index value can take, for example, a value of 0 to 10, and the value of each item determined by the training processing is set to an intermediate value “5”.

For example, the inference parameter for the item “smoothness” is a weight set of each eigenvector in the ASM described above. For example, the inference parameter change unit 36 increases the weight of the high-rank principal component and decreases the weight of the low-rank principal component for the weight of each eigenvector in the ASM as the user indicates a value closer to “0” for the index value of the item “smoothness”, and the inference parameter change unit 36 decreases the weight of the high-rank principal component and increases the weight of the low-rank principal component for the weight of each eigenvector in the ASM as the user indicates a value closer to “10” for the index value of the item “smoothness”.

In addition, the item “outside/inside” is an item for adjusting the position of the contour line OL of the heart. For example, the contour line OL moves inward in a direction in which the volume of the heart (in the example of a left ventricle in FIG. 3) is reduced as the index value of the item “outer/inner” is closer to “0”, and the contour line OL moves outward in a direction in which the volume of the heart is increased as the index value of the item “outside/inside” is closer to “10”.

It should be noted that the inference parameters are parameters adjusted by the training processing such that the inference result regarding the medical image (ultrasound image in the present embodiment) can be output with high accuracy. Therefore, in a case in which the inference parameters are significantly changed, there is also a concern that the learning model 30 cannot output the inference result regarding the medical image with high accuracy. Therefore, the inference parameter change unit 36 may limit a changeable amount of the inference parameters in response to the instruction from the user to a predetermined amount such that the inference accuracy of the learning model 30 is maintained at a certain level of accuracy.

The inference processing unit 34 inputs the target ultrasound image again to the learning model 30 having the inference parameters changed by the inference parameter change unit 36. Then, the inference processing unit 34 outputs the inference result regarding the target ultrasound image based on the output data of the learning model 30 for the input data.

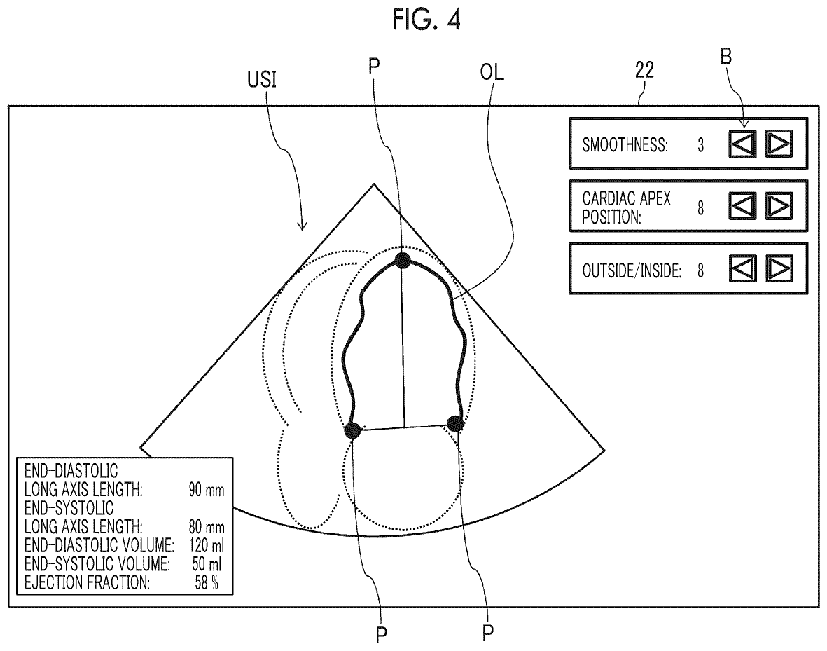

The display controller 20 changes the display content of the inference result of the inference processing unit 34 on the display 22 in response to the change of the inference parameters. FIG. 4 is a diagram showing a display example of the inference result using the changed inference parameters. As a result, the user can bring the inference result regarding the medical image using the learning model 30 close to his/her own request (preference).

The display controller 20 may display the inference result based on the changed inference parameters on the display 22 each time the inference processing unit 34 performs the inference processing based on the changed inference parameters in response to the operation of the button B by the user. As a result, the user can easily obtain the preferred inference result while finely adjusting the inference parameters.

The user may be able to directly make a correction to the inference result (in this example, the contour line OL or the feature position P) regarding the medical image, instead of operating the button B. In this case, the inference parameter change unit 36 may change the inference parameters of the learning model 30 based on the corrected inference result. For example, in a case in which the user manually corrects the shape of the contour line OL, the inference parameter change unit 36 quantifies the smoothness of the contour line OL, and changes the inference parameters (that is, the weight of each eigenvector in the ASM) corresponding to the item “smoothness” based on the quantified smoothness.

In a case in which the inference parameters are changed in response to the instruction from the user, the inference parameter change unit 36 may store the changed inference parameters in the parameter change history DB 32. In particular, in a case in which the inference parameters are changed in response to the instruction from the user, the inference parameter change unit 36 may store a user identifier for uniquely identifying the user who gives the instruction to change the inference parameter and the changed inference parameters in association with each other in the parameter change history DB 32.

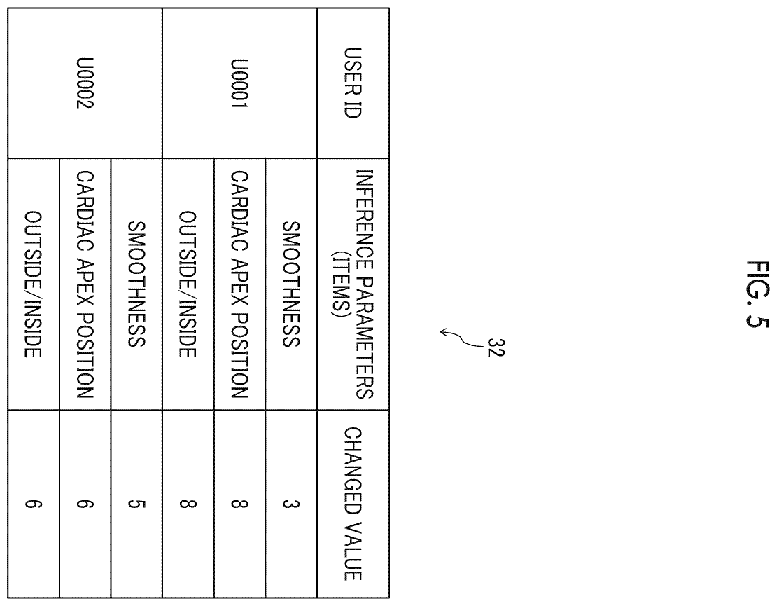

FIG. 5 is a conceptual diagram showing an example of the contents of the parameter change history DB 32. In the present embodiment, a user ID as the user identifier, the inference parameters (items in FIG. 3 or 4), and the index value indicated by the user for the item are stored in the parameter change history DB 32 in association with each other. It should be noted that the user ID of the user can be acquired by the controller 26 by authenticating the user by a method such as a password before the user starts to use the ultrasound diagnostic apparatus 10.

The inference parameter change unit 36 may change the inference parameters of the learning model 30 based on the changed inference parameters stored in the parameter change history DB 32, without depending on the instruction from the user in a case in which the inference processing on the target ultrasound image USI (see FIG. 3 or FIG. 4) is performed in response to the instruction from the user. For example, in a case in which the content of the parameter change history DB 32 is as shown in FIG. 5, the inference parameter change unit 36 determines the inference parameter corresponding to the item “smoothness” of the learning model 30 in the inference of this time automatically, in other words, without depending on the instruction from the user based on the index values “3” and “5” for the item “smoothness” set by the user in the past. For example, the inference parameter change unit 36 first calculates a representative value (for example, an average value) of a plurality of values for the item “smoothness” stored in the parameter change history DB 32. Here, an average value of the plurality of values “3” and “5” for the item “smoothness” is calculated as “4”. Then, the inference parameter change unit 36 changes the inference parameters of the learning model 30 to a value corresponding to the calculated index value “4”. The inference parameter change unit 36 performs the same processing on the other items “cardiac apex position” and “outside/inside”.

In particular, in a case in which the user ID and the changed inference parameters are stored in the parameter change history DB 32 in association with each other, the inference parameter change unit 36 may change the inference parameters of the learning model 30 based on the changed inference parameters associated with the user ID of the user in the parameter change history DB 32 without depending on the instruction from the user in a case in which the inference processing on the target medical image is performed in response to the instruction from the user. For example, in a case in which the content of the parameter change history DB 32 is shown in FIG. 5, the inference parameter change unit 36 changes the inference parameters of the learning model 30 to the values corresponding to the index value “3” for the item “smoothness”, the index value “8” for the item “cardiac apex position”, and the index value “8” for the item “outside/inside” without depending on the instruction from the user in a case in which the inference processing on the target ultrasound image USI is performed in response to the instruction from the user represented by the user ID “U0001”. On the other hand, the inference parameter change unit 36 changes the inference parameters of the learning model 30 to the values corresponding to the index value “5” for the item “smoothness”, the index value “6” for the item “cardiac apex position”, and the index value “6” for the item “outside/inside”, without depending on the instruction from the user, in a case in which the inference processing on the target ultrasound image USI is performed in response to the instruction from the user represented by the user ID “U0002”.

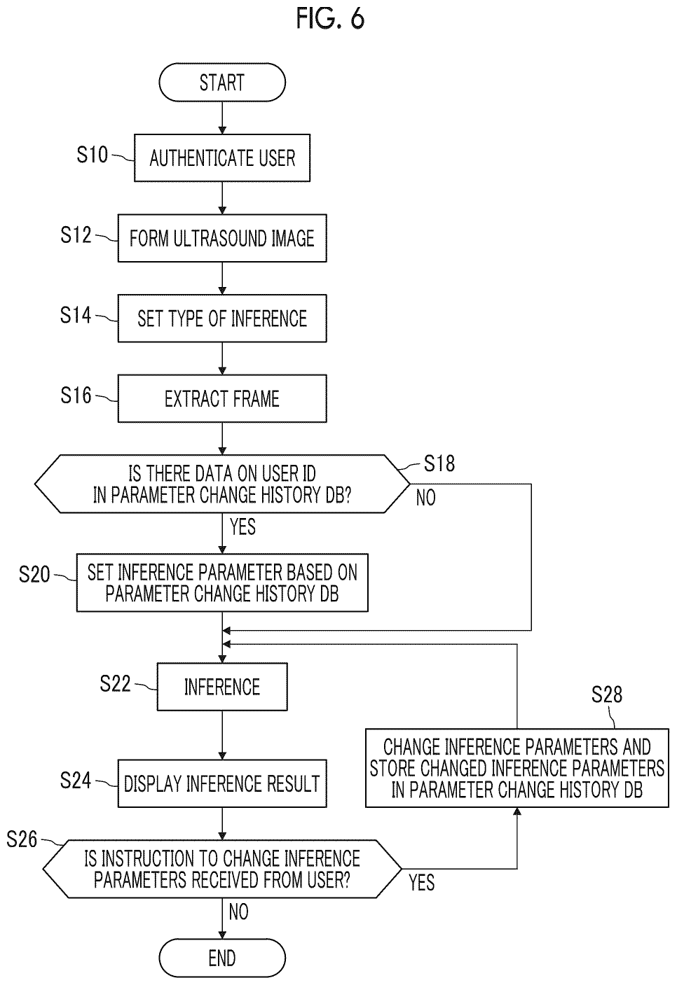

The schematic configuration of the ultrasound diagnostic apparatus 10 according to the present embodiment is as described above. Hereinafter, a flow of the processing of the ultrasound diagnostic apparatus 10 will be described with reference to a flowchart shown in FIG. 6. It should be noted that it is assumed that the learning model 30 has been trained at the start point of the flowchart shown in FIG. 6.

In step S10, the controller 26 authenticates the user to specify the user ID of the user.

In step 512, the image forming unit 18 forms the ultrasound tomographic image based on the reception beam signal subjected to the signal processing by the signal processing unit 16. It is assumed that the four-chamber cross section of the heart is represented in the ultrasound tomographic image.

In step S14, a type of the inference is set by the user. Here, it is assumed that the inference of the contour line and the feature position of the heart is set.

In step S16, a processing target frame (target ultrasound image) is specified by the inference processing unit 34.

In step S18, the inference parameter change unit 36 determines whether or not the data on the user ID specified in step S10 is stored in the parameter change history DB 32. In a case in which the data on the user ID is stored in the parameter change history DB 32, the processing proceeds to step S20, and in a case in which the data on the user ID is not stored, the processing bypasses step S20 and proceeds to step S22.

In step S20, the inference parameter change unit 36 changes the inference parameters of the learning model 30 based on the changed inference parameters associated with the user ID of the user in the parameter change history DB 32.

In step S22, the inference processing unit 34 inputs the target ultrasound image to the learning model 30, and outputs the inference result regarding the target ultrasound image based on the output data of the learning model 30 for the input data.

In step S24, the display controller 20 displays, on the display 22, the inference result of the inference processing unit 34 regarding the target ultrasound image.

In step S26, the inference parameter change unit 36 determines whether or not the instruction to change the inference parameters of the learning model 30 is received from the user.

In a case in which the instruction to change the inference parameter is not received, the processing ends, and in a case in which the instruction to change the inference parameter is received, the processing proceeds to step S28.

In step S28, the inference parameter change unit 36 changes the inference parameters of the learning model 30 in response to the instruction from the user received in step S26. Along with the change, the inference parameter change unit 36 stores the user ID of the user and the changed inference parameters in association with each other in the parameter change history DB 32.

In step S22 after step S28, the inference processing unit 34 inputs the target ultrasound image to the learning model 30 having the inference parameter changed in step S28, and outputs the inference result regarding the target ultrasound image based on the output data of the learning model 30 for the input data.

In step S24 again, the display controller 20 changes the display content of the inference result of the inference processing unit 34 on the display 22 in response to the change of the inference parameters.

Each time the user gives the instruction to change the inference parameter, the processing of steps S22 to S28 is repeated.

Although the embodiment according to the present invention has been described above, the present invention is not limited to the above-described embodiment, and various modifications can be made without departing from the gist of the present invention.

For example, in the above-described embodiment, the medical image processing apparatus is the ultrasound diagnostic apparatus 10, but the medical image processing apparatus is not limited to the ultrasound diagnostic apparatus 10. For example, the medical image processing apparatus may be a personal computer (PC) or a server. In this case, the PC or the server as the medical image processing apparatus includes the display controller 20, the display 22, the input interface 24, the controller 26, the memory 28, the inference processing unit 34, and the inference parameter change unit 36, and the memory 28 stores the learning model 30 and the parameter change history DB 32. The PC or the server as the medical image processing apparatus acquires the target medical image from an X-ray apparatus, a CT apparatus, an MRI apparatus, an ultrasound diagnostic apparatus, or the like, and performs the inference processing on the acquired target medical image.

What is claimed is:

1. A medical image processing apparatus comprising:
a learning model that has been trained to output, based on input data representing a medical image, output data indicating an inference result regarding the medical image;
an inference parameter change unit that changes an inference parameter, which is a parameter that is an adjustment target in training processing of the learning model and that affects the output data, in response to an instruction from a user; and
an inference processing unit that outputs an inference result regarding a target medical image, which is a medical image that is an inference processing target, based on output data in a case in which the target medical image is input to the learning model having the changed inference parameter.

2. The medical image processing apparatus according to claim 1, further comprising:
a display controller that displays the target medical image and the inference result regarding the target medical image on a display, and changes a display content of the inference result on the display in response to the change of the inference parameter.

3. The medical image processing apparatus according to claim 2,
wherein, in a case in which the user makes a correction to the inference result regarding the target medical image displayed on the display, the inference parameter change unit changes the inference parameter based on the corrected inference result.

4. The medical image processing apparatus according to claim 1, further comprising:
a parameter change history database that stores the changed inference parameter that is changed in response to the instruction from the user,
wherein the inference parameter change unit changes the inference parameter based on the changed inference parameter stored in the parameter change history database, without depending on the instruction from the user in a case in which inference processing on the target medical image is performed in response to the instruction from the user.

5. The medical image processing apparatus according to claim 4,
wherein the parameter change history database stores a user identifier for identifying the user who gives the instruction to change the inference parameter and the changed inference parameter that is changed in response to the instruction from the user in association with each other, and
in a case in which the inference parameter related to the user is stored in the parameter change history database, the inference parameter change unit changes the inference parameter based on the changed inference parameter associated with the user identifier of the user in the parameter change history database, without depending on the instruction from the user in a case in which the inference processing on the target medical image is performed in response to the instruction from the user.

6. The medical image processing apparatus according to claim 1,
wherein the medical image is an ultrasound image formed by irradiating a heart of a subject with ultrasound, and
the inference processing unit outputs, as the inference result regarding the target medical image, information used for measurement of the heart using the ultrasound image that is the target medical image.

7. A non-transitory computer readable medium storing instructions that, when executed cause a computer to function as:
a learning model that has been trained to output, based on input data representing a medical image, output data indicating an inference result regarding the medical image;
an inference parameter change unit that changes an inference parameter, which is a parameter that is an adjustment target in training processing of the learning model and that affects the output data, in response to an instruction from a user; and
an inference processing unit that outputs an inference result regarding a target medical image, which is a medical image that is an inference processing target, based on output data in a case in which the target medical image is input to the learning model having the changed inference parameter.
